(19) United States
(12) Patent Application Publication
(10) Pub. No.: US 2011/0201578 A1
(43) Pub. Date: Aug. 18, 2011

(54) COMPOSITION AND METHODS FOR THE TREATMENT OF PSEUDOFOLLICULITIS BARBAE AND SKIN IRRITATION

(76) Inventor: Nyangenya Maniga, Gilbert, AZ (US)

(21) Appl. No.: 13/025,310

(22) Filed: Feb. 11, 2011

Related U.S. Application Data
(60) Provisional application No. 61/304,461, filed on Feb. 14, 2010.
(60) Provisional application No. 61/306,519, filed on Feb. 21, 2010.

(51) Int. Cl. A61K 31/616; A61P 17/00

(52) U.S. Cl. 514/160

(57) ABSTRACT

The invention comprises a composition for the treatment of pseudofolliculitis barbae and a method of use therefor. Preferred embodiments include compositions containing one or more of active ingredients L-arginine, acetylsalicyclic acid, and salicyclic acid adjusted to a pH in the range of 4.0 to 6.0. Preferred compositions include: a carrier, such as water; a moisturizer, such as propylene glycol, and one or more of the active ingredients. Application of a composition for the treatment of pseudofolliculitis barbae with a pH of about skin does not disrupt normal skin functioning.

### CROSS-REFERENCES TO RELATED APPLICATIONS

This application claims:
the benefit of U.S. provisional patent application No. 61/304,461 filed Feb. 14, 2010; and
the benefit of U.S. provisional patent application No. 61/306,519 filed Feb. 21, 2010,
all of which are incorporated herein in their entirety by this reference thereto.

### BACKGROUND OF THE INVENTION

1. Field of the Invention

This invention relates generally to treatment of pseudofolliculitis barbae, skin bumps, and/or skin irritation resulting from waxing and/or shaving.

2. Discussion of the Prior Art

### Pseudofolliculitis Barbae

Pseudofolliculitis barbae (PFB) is a condition commonly referred to as ‘razor bumps’, which most often occurs in the beard area of men of African American, Mediterranean, or Jewish descent and other people with curly hair. The condition is commonly a direct result of shaving. The act of shaving sharpens the end of highly curvy hairs, which grow back into the skin resulting in inflammation and/or a foreign body reaction. Similar results often occur when other areas of the body are shaved, such as under the arm, on the leg, or in the groin area.

### Pseudofolliculitis Barbae Prevention

An effective prevention method for avoidance of pseudofolliculitis barbae is to let the hair or beard grow as once hairs get to be a certain length they will not grow back into the skin. However, clean shaven skin is preferred by many individuals.

### Pseudofolliculitis Barbae Treatment

For treatment of pseudofolliculitis, some men use: shaving powders to avoid the irritation, a single blade razor to avoid shaving too closely, or hair removing techniques, such as electrolysis or laser hair removal to minimize PFB. Still others try shaving every other day, rather than daily, to reduce pseudo-folliculitis barbae. Yet still others soften the beard first with a hot, wet washcloth for five minutes.

Compounds exist for the treatment of pseudofolliculitis barbae. For example, medications used to speed healing of the skin include glycolic acid, antibiotic gels, or oral antibiotics. However, these compounds are different in chemical composition from the present invention. Further, the fact that pseudofolliculitis barbae is still a serious condition is ample evidence that a more effective remedy is needed.

Patents related to the current invention are summarized here.
W. Bliss, “Method for Treating Pseudofolliculitis Barbae”, U.S. Pat. No. 4,228,163 (Oct. 14, 1980) describes a method for treating pseudofolliculitis barbae by topically administering to a person an effective dose of benzoyl peroxide and chlorohydroxyquinoline, and the compositions thereof.
N. Burgess, “Method for the Treatment of Razor Bumps”, U.S. Pat. No. 4,463,016 (Jul. 31, 1984) describes a method of treating razor bumps through administration of a vehicle for topical application of 4-chloro-3,5-diloweralkyl-phenol.
R. Tutsky, “Skin Care and Shaving Composition”, U.S. Pat. No. 4,525,344 (Jun. 25, 1985) describes a shaving composition that essentially contains a phospholipid, wheat germ oil, and vitamin E that prevents pseudofolliculitis.
N. Perricone, “Method for Treatment and Prevention of Pseudofolliculitis Barbae”, U.S. Pat. No. 4,775,530 (Oct. 4, 1988) describes the treatment and prevention of pseudofolliculitis barbae, razor bumps, through topical application to beard areas of an alpha-hydroxy acid or derivative thereof, most preferably glycolic acid or a glycolic acid derivative as a component of a cosmetically acceptable lotion, cream, ointment, soap, shaving foam, stick, gel, or solution.
R. Tutsky, “Skin Care and Shaving Composition”, U.S. Pat. No. 4,784,849 (Nov. 15, 1988) describes a shaving composition that essentially contains a phospholipid, wheat germ oil, and vitamin E that prevents pseudofolliculitis.
W. Crutcher, “Method for the Treatment of Pseudofolliculitis Barbae”, U.S. Pat. No. 4,867,967 (Sep. 19, 1989) describes a method of treating pseudofolliculitis barbae, which includes topically applying a vehicle for delivery of povidone-iodine.
M. Moore, “Shaving Preparation for Treatment and Prevention of PFB (Ingrown Hairs)”, U.S. Pat. No. 4,944,939 (Jul. 31, 1990), describes a shaving preparation aid for treatment of pseudofolliculitis barbae, which includes a composition including one-tenth to six percent by weight salicylic acid, a glucocorticoid in an amount effective to reduce inflammation, and from about one-half to five percent by weight sulfur.
S. Victor, “Shaving Cream Composition for the Treatment of Acne Vulgaris and Pseudofolliculitis Barbae and Method of Producing and Using Same”, U.S. Pat. No. 5,204,093 (Apr. 20, 1993), describes a gel-foam shaving cream composition containing a therapeutically effective amount of benzoyl peroxide used in the topical treatment of pseudofolliculitis barbae.
S. Rosen, et. al., “Topical Agent and Method for the Treatment of Pseudofolliculitis Barbae”, U.S. Pat. No. 5,034,221 (Jul. 23, 1991) describe a topical agent and method for the treatment and prevention of pseudofolliculitis barbae, commonly known as razor bumps, where the composition is a combination of acetylsalicyclic acid, corn starch, isopropyl alcohol, and aloe vera.
M. Burns, “Topical Agent and Method for the Treatment of Pseudofolliculitis Barbae”, U.S. Pat. No. 5,435,997 (Jul. 25, 1995), describes a topical agent and method for the treatment and prevention of pseudofolliculitis barbae, related secondary infections, and other skin conditions associated with shaving using a combination of all of benzoyl peroxide, camphor oil, and aloe present in a 15:4:1 parts by volume ratio in the topical agent composition.
B. Bailey, “Preparation for and Method of Treating Pseudofolliculitis Barbae”, U.S. Pat. No. 5,494,669 (Feb. 27, 1996), describes a method for treating pseudofolliculitis barbae comprising use of a mixture containing alophatic alcohol, liquid aloe, liquid camphor, and the soluble materials of the fresh fig leaves of Ficus Carica.
M. Klein, “Medicinal Facial Mask”, U.S. Pat. No. 5,599,546 (Feb. 4, 1997) describes a class of dermatological compositions for treating skin conditions that include compositions having an acidic material selected from the group consisting of: alphahydroxy acids, carboxylic acids, halocarboxylic acids, dicarboxylic acids, and combinations thereof. The composition is applied to the skin as a facial mask and allowed to dry. The compositions both degrease the skin and deliver therapeutic agents to the skin on a time release basis.
T. McKenzie, “After Shave Treatment composition”, U.S. Pat. No. 5,747,021 (May 5, 1998) describes an after shave treatment composition including at least eleven constituents including acetylsalicyclic acid and a polymer of ethylene oxide.
I. Willis, et. al., “Shaving Composition and Method for Preventing Pseudofolliculitis Barbae”, U.S. Pat. No. 5,853,709 (Dec. 29, 1998) describe a topically applied shaving composition for treatment of a human subject suffering from pseudofolliculitis barbae, the composition comprising: an active ingredient of bacteriostatic/hemostyptic agent, an aqueous or water miscible solvent, a volatile silicone, and a thickening agent.
S. Rosen, et. al., “Topical Compositions and Methods for Treating Pseudofolliculitis Barbae and Ingrown Hair”, U.S. Pat. No. 6,001,340 (Dec. 14, 1999) describe topical compositions and methods for treatment of pseudofolliculitis barbae and ingrown hair. The compositions include: about five percent by weight up to saturation of acetylsalicyclic acid dissolved in a solvent mixture comprising five to fifteen percent by volume propylene glycol, one to ten percent by volume glycerin, and a balance of isopropyl alcohol.
S. Rosen, et. al., “Topical Compositions and Methods for Treating Pseudofolliculitis Barbae and Ingrown Hair”, U.S. Pat. No. 6,156,299 (Dec. 5, 2000) describe topical compositions and methods for treatment of pseudofolliculitis barbae and ingrown hair. The compositions comprise: about five percent by weight up to saturation of acetylsalicyclic acid dissolved in a solvent mixture comprising five to fifteen percent by volume propylene glycol, one to ten percent by volume glycerin, and a balance of isopropyl alcohol.
E. Ita, “After Shave Treatment Preparation”, U.S. Pat. No. 6,277,362 B1 (Aug. 21, 2001) describes a non-steroidal anti-inflammatory after shave formulation to aid in the prevention of pseudofolliculitis barbae, where the formulation comprises: ethanol, salicyclic acid, propylene glycol, glycerin, fragrance, and at least one non-steroidal anti-inflammatory agent.
A. Shabazz, “Composition for the Treatment of Pseudofolliculitis Barbae and Skin Irritation and Method for the Application Thereof”, U.S. Pat. No. 6,352,690 B1 (Mar. 5, 2002) describes a formulation for the treatment of pseudofolliculitis barbae consisting essentially of ten to twenty percent camphor, five percent fragrance oil, and one to two percent mineral oil, and a petroleum balance.
R. Ortiz, et. al., “Therapeutic After-Shave Care Lotion”, U.S. Pat. No. 6,352,691 B1 (Mar. 5, 2002) describe an after shave lotion for use in reducing inflammation and irritation of skin immediately following shaving, where the composition includes: aloe vera gel, vitamin C (ascorbic acid), vitamin A (tocopherol), and hydrocortisone acetate.
G. Mildenberger, et. al., “Non-alcoholic and Hypoallergenic Face Cream for the Treatment of Razor Bumps”, U.S. Pat. No. 6,503,496 B2 (Jan. 7, 2003) describe a medication in liquid form for topical application in the treatment of razor bumps eschewing an alcohol content in favor of water, glycerin, aluminum polyhydrate, propylene glycol, cellulose gum, trisodium ethylenediamine-tetraacetic acid, methylparaben, and imidazolidynl urea.
S. Rosen, et. al., “Topical Compositions and Methods for Treating Pseudofolliculitis Barbae and Ingrown Hair”, U.S. Pat. No. 6,703,009 B1 (Mar. 9, 2004) describe topical compositions and methods for treatment of pseudofolliculitis barbae and ingrown hair. The compositions include: about five percent by weight up to saturation of acetylsalicyclic acid dissolved in a solvent mixture comprising five to fifteen percent by volume propylene glycol, one to ten percent by volume glycerin, and a balance of isopropyl alcohol.
V. Bright-Ellington, “Shaving Preparations and Methods for the Prevention and Treatment of Pseudofolliculitis Barbae”, U.S. Pat. No. 6,843,983 B2 (Jan. 18, 2005) describes shaving prevention methods for the prevention and treatment of pseudofolliculitis barbae with a composition containing glucocorticoid and an antibacterial agent with an optional therapeutic amount of benzoyl peroxide.
A. Khaiat, et. al., “Topical Treatment of Ingrown Hairs”, U.S. Pat. No. 7,252,831 B2 (Aug. 7, 2007) describe compositions and methods for inhibiting the development of ingrown hairs by applying compositions containing sebum reduction agents, keratolytic agents, and anti-inflammatory agents.
R. Norburn, “Shaving, After-Shave, and Skin Conditioning Compositions”, U.S. Pat. No. 7,354,573 B2 (Apr. 8, 2008) describes a skin care composition comprising about twenty percent to about eighty-five percent by weight water, about two percent to about forty percent by weight stearic acid, and about two percent to about forty percent by weight glycerin, diglycerin, grapeseed oil, or a combination thereof.
J. Faryniarz, et. al., “Methods and Compositions Useful to Prevent In-Grown Hair Arising from Shaving”, U.S. Pat. No. 7,404,949 B2 (Jul. 29, 2008) describes a method and composition for managing in-grown hair, which involves applying to those areas malonic acid or a salt thereof in a carrier.

### PROBLEM

There exists in the art a need for a treatment and/or prevention composition and method of application thereof for pseudofolliculitis barbae, which does not irritate the skin, peel skin, or alter the pH of skin and its corresponding functionality.

### SUMMARY OF THE INVENTION

The invention comprises a composition for the treatment of pseudofolliculitis barbae and method for the application thereof.

### DESCRIPTION OF THE FIGURES

FIG. 1 illustrates a first example of pseudofolliculitis barbae;

FIG. 2 illustrates a second example of pseudofolliculitis barbae; and

FIG. 3 illustrates various forms of L-arginine.

### DETAILED DESCRIPTION OF THE INVENTION

The invention comprises a composition for the treatment of pseudofolliculitis barbae and method for the application thereof. Various embodiments of the composition are described, infra. In use, all or part of the composition is applied to the skin surface and optionally enters into the skin and/or penetrates into a hair follicle, pore, wound, pustule, and/or papule. The composition is optionally pH balanced so as to not irritate the skin or alter its functionality.

Pseudofolliculitis barbae, PFB, is a medical term for persistent irritation caused by shaving. Pseudofolliculitis is also colloquially referred to as “razor bumps”. Pseudofolliculitis is most common on the male face, but it also results on other parts of the male or female body where hair is shaved or plucked, especially areas where hair is curly and the skin is sensitive, such as in genital shaving, which is also referred to as PFB and more specifically as pseudofolliculitis pubis.

Curly hair tends to curl into the skin instead of straight out the follicle and in some instances pierces the skin leading to any of: an inflammation reaction, itchiness, redness, an creation of pimples. In some instances, the inflamed regions result in papules or pustules especially if an infection results. Papules and pustules are specific examples of pseudofolliculitis barbae or outcomes of pseudofolliculitis barbae.

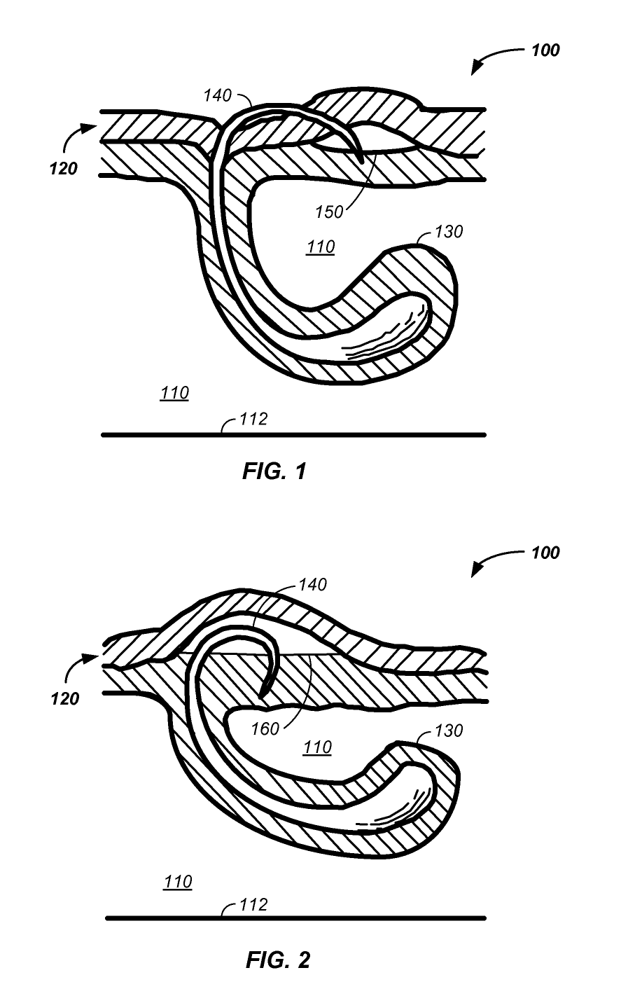

Referring now to FIGS. 1 and 2, skin 100 is illustrated. The skin 100 contains a dermis layer 110, which resides above subcutaneous tissue 112 and below the epidermis 120. The epidermis 120 is an outer layer of the skin residing just below the stratum corneum. A hair follicle 130 is a skin component that grows hair by packing old cells together.

Referring now to FIG. 1, a first type of pseudo folliculitis barbae resulting from extrafollicular hair is illustrated. Extrafollicular hair is hair that has exited the follicle and reentered the skin. As illustrated, the hair 140 has grown back into the skin 100 forming an infected area or papule 150. A papule is an about solid elevation of skin with no visible fluid varying in size from a pinhead to about one centimeter. A papule may appear pink, red, or brown in coloration.

Referring now to FIG. 2, a second type of pseudo folliculitis barbae resulting from transfollicular hair is illustrated. Transfollicular hair never exits the follicle, but because of its naturally curly nature curls back into the follicle causing fluid build-up and irritation. The hair follicle 130 is observed to have formed a pustule 160, which is a small elevation of skin that contains any of purulent material, necrotic inflammatory cells, and/or a collection of neutrophils.

In one embodiment, a composition is used for prevention of and/or treatment of pseudofolliculitis barbae or skin bumps. A preferred composition includes L-arginine with any of aspirin or acetylsalicyclic acid or salicyclic acid. Additional optional compositions include L-arginine with any of:
one or more hydroxyacids; and
a mixture of one or more hydroxyacids and aspirin.
   In any of the compositions, water is an optional carrier or diluent. An additional optional composition element is a skin moisturizer, such as propylene glycol.

In another embodiment, an the composition includes: at least one amino acid and at least one of α-hydroxy acid, salicyclic acid, and acetyl salicyclic acid. While any amino acid is optionally used, the amino acids of arginine, lysine, and/or histidine are preferred as they are of a basic pH and do not result in deproteinization of the aforementioned acids. The hydrogen ion is held in a complex with the amino acid. By not removing the proton, the acid is intact and more readily penetrates the skin. Additionally, the acid pH of the amino acids does not result in a free proton, which is otherwise corrosive to the skin. Lysine, histidine, and arginine are preferred, but any protein is optionally used and hydrolyzed. For example, collagen or protein is used as a composition component and is hydrolyzed in the composition to result in amino acids, which react as described herein with one or more of α-hydroxy acid, salicyclic acid, and acetyl salicyclic acid.

In yet another embodiment, the composition includes at least one amino acid; at least one of α-hydroxy acid, salicyclic acid, and acetyl salicyclic acid; and a base.

### Composition Ingredients

In yet another embodiment of the invention, the composition contains one of and preferably both of L-arginine and acetylsalicyclic acid and at least one of or any combination of: water, aloe vera gel, propylene glycol, lecithin, lactic acid, an alpha-hydroxy acid, shea butter, a lipid, a lipid material, retinoic acid, ascorbic acid, xanthan gum, and/or hydroxypropylmethyl cellulose.

### L-Arginine

A first optional active ingredient, active material, or active element of the composition is L-arginine, an enantiomer of arginine, and/or arginine, with a concentration range of 0.1 to 40 percent. L-arginine is an essential amino acid, C₆H₁₄N₄O₂. The recommended daily intake of L-arginine is two grams per day. The free amino acid is also generated by hydrolysis or digestion of plant and/or animal protein. L-arginine is a vasodilator than increased blood flow, is a fat solubilizer, and cures wounds. The free amino acid increases insulin secretion and is converted to urea in the liver by arginase.

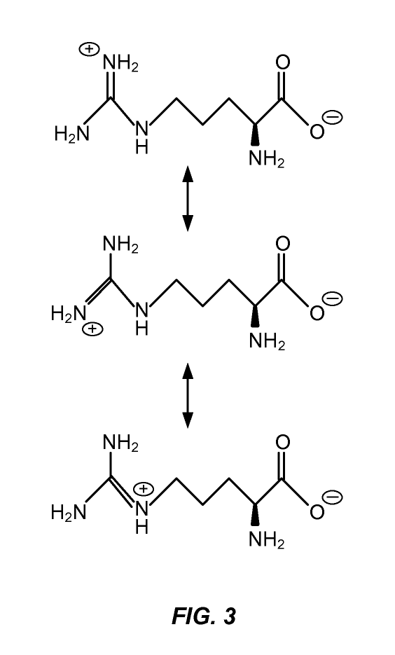

The amino acid side chain of arginine includes a 3-carbon aliphatic straight chain, the distal end of which is capped by a complex guanidinium group. With a pKₐ of 12.48, the guanidinium group is positively charged in neutral, acidic and even most basic environments, and thus imparts basic chemical properties to arginine. Because of the conjugation between the double bond and the nitrogen lone pairs, the positive charge is delocalized, enabling the formation of multiple hydrogen bonds, H-bonds. Referring now to FIG. 3, L-arginine is illustrated in various forms showing the delocalization of charge in the guanidinium group.

Arginine plays an important role in cell division, the healing of wounds, removing ammonia from the body, immune function, and the release of hormones. The benefits and functions attributed to oral supplementation of L-arginine include:
use as a precursor for the synthesis of nitric oxide, (NO);
reduces healing time of injuries;
vasodilatation;
quickens repair time of damaged tissue; and
helps decrease blood pressure.
   As a precursor, arginine is used in generation of nitric oxide, which relaxes blood vessels and is used when vasodilation is required.

In another embodiment, any of: L-arginine; arginine; lysine; glycine; histidine; a pH basic amino acid, such as an amino acid having at least one pKa greater than 7.0; and/or a fat solubilizer is used as an active ingredient in the formulation or composition used in the treatment of pseudofolliculitis barbae. The percentage of L-arginine, lysine, and/or glycine alone or in combination is preferably about 0.1, 5, 10, 15, 20, 25, 30, 35, or 40 percent by volume or mass. Optionally, any source of amino acids is used, such as hydrolysed collagen or a protein hydrolysate. Optionally glucosamine and/or n-acetylglucosamine are used to dampen, adjust, or buffer the pH of the resulting composition.

### Acetylsalicyclic Acid

A second optional active ingredient, active material, or active element of the composition is acetylsalicyclic acid, with a concentration range of 0.1 to 40 percent. Acetylsalicyclic acid and its metabolized form of salicyclic acid each decrease inflammation. Addition of the acetyl group to salicyclic acid results in a molecule that is more non-polar than salicyclic acid. The more polar acetylsalicyclic acid is more readily absorbed into skin cells and more readily transports across a lipid membrane of a skin cell. Once inside the cell, the acetyl group is removed to form an active ingredient of salicyclic acid. Hence, acetylsalicyclic acid is a preferred constituent of the composition for treating pseudofolliculitis barbae. However, an optional embodiment of the composition for treatment of PFB contains one or more of: salicyclic acid and/or any α-hydroxy acid, in the absence of acetylsalicyclic acid.

The inventor has determined that used by itself, acetylsalicyclic acid acts as a skin peeler, as the pKa is about 1. The low pKa results in an acidic solution having a pH of about 1.9, which damages skin and results in skin peeling in the hours and/or days following application to skin. Particularly, application of acetylsalicyclic acid in a composition having a pH of about 3.8 or less results in one or more of the detrimental effects of: an acid burn of the skin, inflammation, itching, sensitizing, dermatitis, and/or skin peeling. The inventor discovered that adjusting the pH of a composition used to treat pseudofolliculitis barbae containing acetylsalicyclic acid or salicyclic acid to about 5.5 or about 4.0 to 6.5 reduces and/or eliminates the above described detrimental effects.

In another embodiment, any of acetylsalicyclic acid, salicyclic acid, and or α-hydroxy acid is used as an active ingredient in the formulation or composition used in the treatment of pseudofolliculitis barbae. The percentage of acetylsalicyclic acid, salicyclic acid, and or α-hydroxy acid alone or in combination is preferably about 0.1, 5, 10, 15, 20, 25, 30, 35, or 40 percent by volume or mass.

### Optional Active Elements

L-arginine and acetylsalicyclic acid are preferred active ingredients, constituents, or elements of a composition or formulation for the treatment of pseudofolliculitis barbae. Additional or substitute optional active elements are described herein. One optional active element is retinoic acid. A preferred concentration range of retinoic acid is 0 to 0.05 percent. Retinoic acid is an oxidized from of Vitamin A and contains only partial functionality of Vitamin A. Retinoic acid acts faster on pseudofolliculitis barbae as the acid is more non-polar than Vitamin A and hence transports across, into, or through skin layers more efficiently. An additional optional active element is Vitamin A. Both retinoic acid and Vitamin A remove swelling and/or remove dead skin. Yet another optional active element is lactic acid, which is an α-hydroxy acid. Lactic acid is a preservative, which is optionally used in place of and/or in combination with aspirin and/or acetylsalicyclic acid in the composition. Still yet additional active elements include any of: lysine, histidine, hydrolysed collagen, and protein hydrolysate.

### Water

Water is an optional component of the composition. Water is a carrier and/or diluent. When used, the percentage of water is preferably about 20 to 99 percent and more preferably about 40, 50, 60, 61, 62, 63, 64, 65, 70, or 80 percent by volume or mass.

### Additional Components

Each of aloe vera gel, propylene glycol, lecithin, shea butter, or a solubilizer are both (1) optional composition components and (2) perform as skin moisturizers. Shea butter, lecithin, and/or a lipid material is optionally used independently or in combination to enhance penetration of the active material, such as L-arginine and/or acetylsalicyclic acid, into the skin 100. Propylene glycol additionally performs as a penetration enhancer and performs as a solubilizer for salicyclic acid and/or acetylsalicyclic acid. The percentage of aloe vera gel, propylene glycol, lecithin, shea butter, or a solubilizer alone or in combination is preferably about 0, 5, 10, 15, 20, 25, 30, 35, or 40 percent by volume or mass.

Ascorbic acid is another optional composition constituent, which performs as an anti-oxident. Ascorbic acid is a water soluble sugar acid with antioxidant properties. One form of ascorbic acid is commonly known as vitamin C. In another embodiment, ascorbic acid is optionally used in vitamin A, as an anti-oxidant, in a composition used to treat pseudofolliculitis barbae. Additional optional composition elements include each of xanthan gum and hydroxypropylmethyl cellulose, which perform as composition thickeners. Further, the xanthan gum performs as a rheology modifier.

pH

The pH of skin is about 5.5. When the pH of the skin surface is less than about 3.8, skin peeling results. Additionally, side effects of skin adjusted to a pH of less than about 3.8 include: skin inflammation, dermatitis, itching, and/or sensitizing of the skin.

The inventor has determined that, in still yet another embodiment, a preferred pH range of the composition is in the range of about 4.0 to 6.0, which prevents the above described skin inflammation, dermatitis, itching, and/or sensitizing of the skin. A more preferred pH of the composition or formulation for the treatment of pseudofolliculitis barbae is about 4.5, 5.0, or 5.5.

A preferred composition or formulation for the treatment of pseudofolliculitis barbae is provided in Table 1. The combination results in a pH in the range of 4.0 to 6.0. For example, the pKa of L-arginine of 12.48 adjusts the pH of the acetylsalicyclic acid up into the desired pH range of 4.0 to 6.0 and prevents the acid peeling resulting from the unbuffered and/or non pH adjusted acetylsalicyclic acid. Further, the application of a composition for the treatment of pseudofolliculitis barbae with a pH of about skin does not disrupt normal skin functioning.

TABLE 1
Preferred pseudofolliculitis barbae treatment formulation

| Ingredient | Percentage | Purpose |
|---|---|---|
| water | 60 to 64 | diluent |
| propylene glycol | 18 to 22 | skin moisturizer |
| acetylsalicyclic acid | 7 to 11 | active ingredient |
| L-arginine | 7 to 11 | active ingredient |

In another embodiment, a formulation or composition for the treatment of pseudofolliculitis barbae is provided. The combination results in a pH in the range of 4.0 to 6.0, includes a carrier, active ingredient, solubilizer, and skin moisturizer. The preferred formulation is provided in Table 1. However, each of the constituents present in Table 1 is optionally present in a range of concentrations. In another embodiment, the formulation contains: water in the range of about 20 to 99 percent, acetylsalicyclic acid in the range of about 0.1 to 40 percent, L-arginine in the range of about 0.1 to 40 percent, and propylene glycol in the range of about 0 to 40 percent, where percent is by either volume or mass. For example, the concentration of a first active ingredient of L-arginine is preferably about 9 percent; however a range of percentages of about 1, 2, 3, 4, 5, 6, 7, 8, 9, or 10 percent are acceptable and the concentration of a second active ingredient of acetylsalicyclic acid is preferably about 9 percent; however a range of percentages of about 0.5, 1, 2, 3, 4, 5, 6, 7, 8, 9, or 10 percent are acceptable, with the balance of the water and propylene glycol adjusted. The percentage of water is preferably about 59, 60, 61, 62, 63, 64, or 65 percent. The percentage of propylene glycol is preferably about 18, 19, 20, 21, or 22 percent.

In yet another embodiment, a formulation or composition for the treatment of pseudofolliculitis barbae is provided in Table 2.

TABLE 2
Formulation for treatment of
pseudofolliculitis barbae

| Ingredient | Percentage |
|---|---|
| water | 20-99 |
| propylene glycol and/or solubilizer | 0 to 40 |
| acetylsalicyclic acid, salicyclic | 0.1 to 40 |
| acid, and/or α-hydroxy acid | |
| L-arginine, lysine, and/or glycine | 0.1 to 40 |
| pH | 4.5 to 6.0 |

In still yet another embodiment, a formulation or composition or formulation for the treatment of pseudofolliculitis barbae is provided in Table 3. Percentages are optionally by mass or by volume. The formulation is optionally any combination of elements listed in Table 3 with a pH 4.0 to 6.0 or an adjusted pH of 4.0 to 6.0.

TABLE 3
Formulation for treatment of pseudofolliculitis barbae

| Ingredient | Percentage |
|---|---|
| water and/or a diluent | 20 to 99 |
| aloe vera gel | 0 to 99 |
| propylene glycol and/or a moisturizer | 0 to 50 |
| acetylsalicyclic acid, salicyclic acid, and/or | 0.1 to 40 |
| a α-hydroxy acid | |
| lecithin | 0 to 20 |
| L-arginine, lysine, and/or glycine | 0.1 to 40 |
| lactic acid | 0 to 20 |
| shea butter | 0 to 20 |
| retinoic acid | 0 to 0.5 |
| ascorbic acid and/or an anti-oxidant | 0 to 5 |
| xanthan gum | 0 to 3 |
| hydroxypropylmethyl cellulose and/or a thickener | 0 to 3 |

In yet still another embodiment, the composition contains at least aspirin adjusted to a pH of about 4.0 to 6.0.

### Method of Treatment

The formulation or composition is applied through a method of application of a thin veneer of the formulation to the shaved area. The shaved area is any of a beard area and any other areas of the body that are shaved, such as under the arm, the leg, or in the groin area. The formulation is applied immediately before shaving, during shaving, after shaving, and/or between shaves to any shaved region or region to be shaved. The formulation is optionally applied as a component of a cosmetically acceptable lotion, cream, ointment, soap, shaving foam, stick, gel, or solution. As skin bumps may result from ingrown hair in the absence of shaving, the formulation or composition for the treatment of pseudofolliculitis barbae and/or skin bumps includes use in the absence of shaving.

Preferably, the formulation is sufficiently viscous to allow the formulation to remain on the affected area of the skin for a sufficient time period to reduce the pseudofolliculitis inflammation and/or to allow partial absorption of the composition into the skin.

In yet still another embodiment, any combination or permutation of any of the above described constituents is used to form a composition for the treatment of bromhidrosis.

In still yet another embodiment, any combination or permutation of any of the above described constituents is used to form a formulation, composition, and/or mixture, cream, lather, foam, ointment, soap, liquid, or gel for application to the skin for the treatment of pseudofolliculitis barbae and/or skin bumps.

Although the invention has been described herein with reference to certain preferred embodiments, one skilled in the art will readily appreciate that other applications may be substituted for those set forth herein without departing from the spirit and scope of the present invention. Accordingly, the invention should only be limited by the Claims included below.

1. A composition for the treatment and prevention of pseudofolliculitis barbae, comprising:
at least one amino acid comprising a net concentration of about one-tenth to forty percent by weight of said composition; and
at least one of:
about one-tenth to forty percent α-hydroxy acid, by said weight of said composition;
about one-tenth to forty percent acetylsalicyclic acid, by said weight of said composition; and
about one-tenth to forty percent salicyclic acid, by said weight of said composition,
wherein said composition is configured for treatment and prevention of pseudofolliculitis barbae, and
wherein the composition comprises a form of any of:
a spray;
a solid stick;
a soft solid;
a cream; and
a gel.

2. The composition of claim 1, wherein said at least one amino acid comprises a hydrolysis component of at least one of:
a collagen; and
a protein.

3. The composition of claim 1, wherein said at least one amino acid comprises at least one of:
arginine;
lysine;
histidine; and
glycine.

4. The composition of claim 3, said arginine comprising L-arginine.

5. The composition of claim 3, said composition further comprising:
a base; and
a pH of about 4.0 to 6.0.

6. The composition of claim 5, said pH comprising about 5.4 to 5.6.

7. The composition of claim 3, said at least one amino acid comprising:
a basic amino acid comprising at least one pKa greater than about 7.0.

8. The composition of claim 3, further comprising at least one of:
glucosamine; and
n-acetylglucosamine.

9. The composition of claim 5, further comprising:
a skin moisturizer, said skin moisturizer comprising at least one of:
glycerin;
aloe vera gel;
propylene glycol;
lecithin;
shea butter;
a lipid; and
a lipid material,
wherein said skin moisturizer comprises about one-tenth to forty percent, by said weight, of said composition

10. The composition of claim 5, said composition further comprising:
water.

11. The composition of claim 10, wherein said water comprises a concentration of about sixty to sixty-five percent, by said weight of said composition.

12. The composition of claim 10, wherein said α-hydroxy acid comprises lactic acid.

13. The composition of claim 10, further comprising:
an antioxidant, comprising at least one of
about one-tenth to five percent ascorbic acid, by said weight of said composition;
about one-tenth to five percent vitamin A, by said weight of said composition;
about one-tenth to five percent vitamin E, by said weight of said composition; and
about 0.01 to 0.05 percent retinoic acid, by said weight of said composition.

14. The composition of claim 10, further comprising:
a thickener, comprising at least one of:
xanthan gum; and
a hydroxypropylmethyl cellulose.

15. The composition of claim 1, further comprising:
a rheology modifier.

16. The composition of claim 1, further comprising:
about sixty to sixty-four percent water, by said weight of said composition; and
at least one of:
about one to twenty-two percent glycerin, by said weight of said composition,
about one to twenty-two percent propylene glycol, by said weight of said composition,
wherein said acetylsalicyclic acid is present in a concentration of about seven to eleven percent, by said weight of said composition, and
wherein said at least one amino acid comprises L-arginine, said L-arginine comprising about seven to eleven percent, by weight, of said composition.

17. A method for using a composition for the treatment and prevention of pseudofolliculitis barbae, comprising the steps of:
providing in said composition at least one amino acid comprising a net concentration of about one-tenth to forty percent by weight of said composition; and
providing in said composition at least one of:
an α-hydroxy acid;
about one-tenth to forty percent acetylsalicyclic acid; and
about one-tenth to forty percent salicyclic acid; and
applying said composition as a thin veneer to a shaving area.

18. The method of claim 17, further comprising the steps of:
providing at least one of:
a collagen; and
a protein; and
hydrolyzing in the composition at least one of said collagen and said protein to form said amino acid.

19. The method of claim 17, said composition further comprising:
a base;
a pH of about 4.0 to 6.0;
a skin moisturizer;
an antioxidant; and
a thickener.

20. The method of claim 17, said composition comprising:
about sixty to sixty-four percent water, by said weight of said composition; and
at least one of:
about one to twenty-two percent glycerin, by weight of said composition; and
about one to twenty-two percent propylene glycol, by weight of said composition,
wherein said acetylsalicyclic acid is present in a concentration of about seven to eleven percent, by weight of said composition, and
wherein said at least one amino acid comprises L-arginine, said L-arginine comprising about seven to eleven percent, by weight, of said composition.
